(19) United States
(12) Patent Application Publication
(10) Pub. No.: US 2020/0069945 A1
(43) Pub. Date: Mar. 5, 2020

(54) HIGH DENSITY RETINAL PROSTHESIS SYSTEM WITH EQUALIZATION

(71) Applicant: IRIDIUM MEDICAL TECHNOLOGY CO., LTD., Hsinchu (TW)

(72) Inventors: LONG-SHENG FAN, Hsinchu (TW); FENG-HSIUNG HSU, Hsinchu (TW); YUNG-CHAN CHEN, Hsinchu (TW)

(21) Appl. No.: 16/552,520

(22) Filed: Aug. 27, 2019

Related U.S. Application Data
(60) Provisional application No. 62/724,225, filed on Aug. 29, 2018.

(51) Int. Cl. A61N 1/36; A61N 1/05; A61F 2/14

(52) U.S. Cl. CPC A61N 1/36046; A61F 9/0017; A61F 2/14; A61N 1/0543

(57) ABSTRACT

The present invention discloses a high density retinal prosthesis system with equalization comprises a retinal prosthesis chip, a measuring device, a tuning device, and a transmitting device. After the measuring device capable of generating electrical induction with the retinal prosthesis chip obtains a degree of light stimulation received by pixel units in the retinal prosthesis chip, it is equalized by the tuning device, and then a calibration signal is fed to the pixel units to tune the pixel units to make the pixel units in the retinal prosthesis system to achieve an equalization effect.

### FIELD OF THE INVENTION

The present invention relates to a system for applying to a retinal prosthesis, and more particularly to a high density retinal prosthesis system with equalization.

### BACKGROUND OF THE INVENTION

Currently, among the patients with visual deterioration, some of the patients choose to implant an artificial retina to improve their vision. At present, expensive artificial retinas of the commercial standard with low density pixels have a limited improvement on the quality of life of the patients. In view of this, many companies as well as academic and research institutes have begun to actively invest in the improvement of microsystem for artificial retina.

U.S. Pat. No. 9,155,881B2 filed by the same applicant proposed a non-planar chip set with a flexible structure formed by a curved deformation of a planar shape. The flexible structure comprises at least one semiconductor material layer, around a central portion of the flexible structure, there is a plurality of slit passage openings extending from a periphery of the flexible structure toward the central portion, and the slit passages are used to reduce a displacement stress generated after the planar shape is crookedly deformed to become the flexible structure. Outside the flexible structure, a bonding structure is combined with at least one fixing structure to maintain the flexible structure in a curved state.

Among the many aspects of improving the performance of artificial retina, improving the effective pixels number of the artificial retina is an important topic for discussion. The increase in the pixels number of the artificial retina represents that the number of microelectrodes will increase from past dozens and hundreds to thousands. Further, with the technology getting more mature gradually, many problems that have been seldom considered in the past are getting more and more important.

For example, there are differences or defects between the individual pixels, which may cause unevenness. On the other hand, because the shape of the user's eyeball is not uniform, or the middle and the edges of the eyeball are different, the pixels in different areas output different degrees of stimulation. In order to allow the user to get the most comfortable wearing experience from the beginning, it is theoretically necessary to make tuning and setting for the microelectrodes according to the microenvironment in which the microelectrodes are implanted. In a related prior art, such as U.S. Pat. No. 7,738,962 B2, different stimulations of currents, voltages, pulse durations, or frequencies are applied to the artificial retina, and the responses of the artificial retina to the above-mentioned stimulations are recorded for establishing the individual tuning formulas for the different areas of the artificial retina, and the tuning formulas are stored to be applied in future stimulation to solve the problem that individual patients have different perceptions of the electrical nerve stimulation of the artificial retina.

However, it is expected that the above tuning will be a costly method, and a more economical and convenient system or method is required to efficiently perform the above microelectrode tuning.

### SUMMARY OF THE INVENTION

A main object of the present invention is to solve the problem of poor visual sense generated in the patient that is easily caused by the conventional artificial retina due to factors of differences or defects between the individual pixels, the non-uniform shape of the user's eyeball, or the difference between the middle and the edges of the user's eyeball.

In order to achieve the above object, a retinal prosthesis system is provided. The retinal prosthesis system comprises a retinal implant device, comprising a plurality of pixel units to generate an electro-stimulation signal to stimulate retina neuron cells; a measuring device, measuring an induced electrical potential of the electro-stimulation signal from the retinal implant device and sending a measured signal associated with the induced electrical potential of the plurality of pixel units; a tuning device, electrically connecting to the measuring device, the tuning device receiving the measured signal and performing an equalization process on the measured signal then sending a calibration setting command and tuning parameters according to the equalization process; and a transmitting device, transmitting a calibration signal to the retinal prosthesis chip, wherein the calibration signal is associated with the calibration setting command and the tuning parameters such that a uniformity of the electro-stimulation signals output from a group of the pixel units is improved.

In one embodiment of the present invention, the electro-stimulation signal is generated according to a light stimulus received by the plurality of pixel units.

In one embodiment of the present invention, the electro-stimulation signal is generated according to a command received by the plurality of pixel units.

In one embodiment of the present invention, the retinal implant device further comprising a first induction coil and an electrical connection portion electrically connected to the pixel units.

In one embodiment of the present invention, each of the plurality of pixel units is correspondingly disposed with a storage element storing the tuning parameters.

In order to achieve the above object, a retinal prosthesis system is further provided. The retinal prosthesis system comprises a retinal implant device, the retinal implant device comprising a plurality of pixel units, at least one measuring component, and a transmitting component, the plurality of pixel units generating an electro-stimulation signal to stimulate retina neuron cell, the at least one measuring component measuring an induced electrical potential of the electro-stimulation signal from the pixel units and sending a measured signal associated with the induced electrical potential of the plurality of pixel units through the transmitting component; a tuning device, receiving the measured signal and performing an equalization process on the measured signal then sending a calibration setting command and tuning parameters according to the equalization process; and a transmitting device, transmitting a calibration signal to the retinal implant device, wherein the calibration signal is associated with the calibration setting command and the tuning parameters such that a uniformity of the electro-stimulation signals output from a group of the pixel units is improved.

In one embodiment of the present invention, the electro-stimulation signal is generated according to a light stimulus received by the plurality of pixel units.

In one embodiment of the present invention, wherein the electro-stimulation signal is generated according to a command received by the plurality of pixel units.

In one embodiment of the present invention, wherein each of the plurality of pixel units is correspondingly disposed with a storage element storing the tuning parameters.

In one embodiment of the present invention, wherein the plurality of pixel units and the at least one measuring component are integrated in a retinal prosthesis chip disposed on a sub-retina.

In one embodiment of the present invention, wherein the plurality of pixel units, the at least one measuring component and the transmitting component are integrated in a retinal prosthesis chip disposed on a sub-retina.

In order to achieve the above object, a retinal implant device is further provided. The retinal implant device comprises a plurality of pixel units, generating an electro-stimulation signal to stimulate retina neuron cell; at least one measuring component, measuring an induced electrical potential of the electro-stimulation signal from the pixel units and sending a measured signal associated with the induced electrical potential of the plurality of pixel units; and at least one tuning component, receiving the measured signal and performing an equalization process on the measured signal then generating a calibration setting command and tuning parameters according to the equalization process, wherein a calibration signal associated with the calibration setting command and the tuning parameters is sent to the pixel units such that a uniformity of the electro-stimulation signals output from a group of the pixel units is improved.

In one embodiment of the present invention, the electro-stimulation signal is generated according to a light stimulus received by the plurality of pixel units.

In one embodiment of the present invention, the electro-stimulation signal is generated according to a command received by the plurality of pixel units.

Therefore, the retinal prosthesis system of the present invention performs the equalization process through the tuning device after measuring the stimulation signals outputted by the pixel units in the retinal prosthesis chip, and then returns the calibration signal to the pixel units for tuning the pixel units to avoid the pixel units generating undesirable phenomena such as visual blur which may cause by the unexpected stimulation response of the pixel units due to defects, or the incompatibility of the pixel units with the microenvironment in which the pixel units are located. The equalization process makes uniform electro-stimulation output from each electrode of pixel array given a uniform light intensity or a setting representing a uniform light intensity over the pixel units.

In contrast to the prior art tuning method of U.S. Pat. No. 7,738,962 B2, in which the retinal prosthesis chip must be specially subjected to additional stimulations of different currents, voltages, pulse durations, frequencies to generate the tuning formula, and only the user's subjective feeling is involved during the evaluation process; while the present invention allows tuning in an objective manner by the feedback calibration signal in normal use, and the method is faster and more accurate in tuning leaving user feedback to a later stage of pixel-group fitting.

### BRIEF DESCRIPTION OF THE DRAWINGS

FIG. 1 is a schematic diagram of a retinal prosthesis system according to an embodiment of the present invention;

FIG. 2 is a schematic diagram of a retinal implant device according to an embodiment of the present invention;

FIG. 3 is a schematic diagram of a measuring device according to an embodiment of the present invention;

FIG. 4 is a schematic diagram of a transmitting device according to an embodiment of the present invention;

FIG. 5 is a configuration of pixel units on a retinal prosthesis chip and electroretinogram according to an embodiment of the present invention;

FIG. 6 is a schematic diagram of a retinal prosthesis system according to another further embodiment of the present invention;

FIG. 7 is a schematic diagram of the pixel unit according to an embodiment of the present invention;

FIG. 8 is a schematic diagram of the pixel unit according to another embodiment of the present invention;

FIG. 9 is a schematic diagram of the pixel unit according to a further embodiment of the present invention; and

FIG. 10 is a schematic diagram of a retinal implant device according to a further embodiment of the present invention.

### DETAILED DESCRIPTION OF THE PREFERRED EMBODIMENTS

The detailed description and technical content of the present invention will now be described with reference to the accompanying drawings as follows.

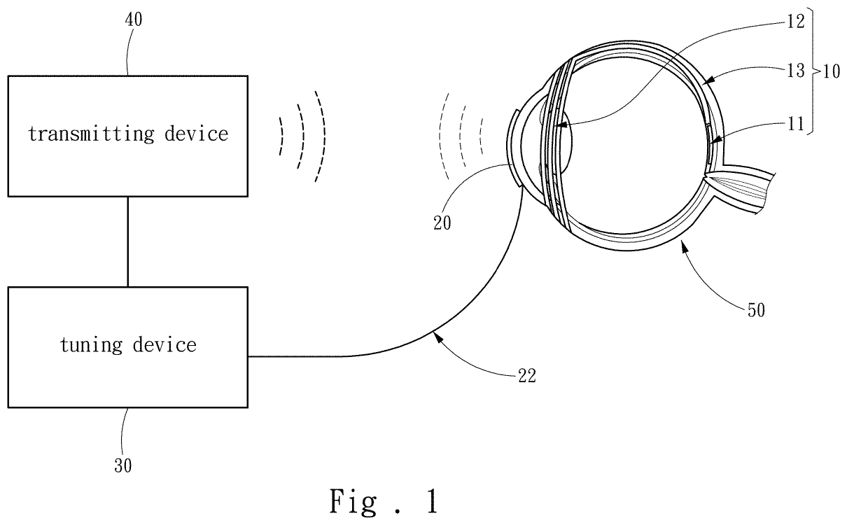

FIG. 1 is a schematic diagram of a retinal prosthesis system according to an embodiment of the present invention, the retinal prosthesis system comprises a retinal implant device 10, a measuring device 20, a tuning device 30, and a transmitting device 40, wherein the retinal implant device 10 comprises a retinal prosthesis chip 11, a first induction coil 12, and an electrical connection portion 13 electrically connected between the retinal prosthesis chip 11 and the first induction coil 12. The retinal implant device 10 and the measuring device 20 are disposed inside an eye structure 50 of a user, and the tuning device 30 and the transmitting device 40 are disposed outside the eye structure 50 of the user, and electrically connected to or electrically induced with the retinal prosthesis chip 11 and/or the measuring device 20 in a wired or wireless manner.

In detail, the retinal prosthesis chip 11 can be disposed on an epi-retina or a sub-retina of the eye structure 50. In this embodiment, the retinal prosthesis chip 11 is disposed on the sub-retina. The retinal prosthesis chip 11 comprises a plurality of pixel units. The retinal prosthesis chip 11 may comprise thousands or even more of the pixel units. The architecture of the retinal prosthesis chip 11 will be described later.

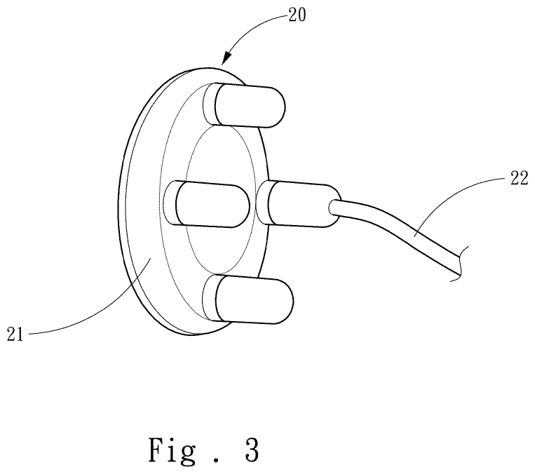

The measuring device 20 can be disposed on a cornea of the user. In one embodiment, the measuring device 20 is in contact with the eye structure 50. Please refer to FIG. 1 and FIG. 3, the measuring device 20 of one embodiment of the present invention comprises a signal receiving and transmitting module 21, and the signal receiving and transmitting module 21 is disposed in a base structure similar to a contact lens and comprises at least one electroretinogram (ERG) electrode. For example, the ERG electrode could be a corneal electrode. After the pixel units of the retinal prosthesis chip 11 receive either light or command to generate an electro-stimulation signal, the measuring device 20 measures an induced electrical potential of the electro-stimulation signal from the retinal prosthesis chip 11. In this embodiment, the ERG electrode measures the induced electrical potential of the electro-stimulation signal from at least one electrode in the retinal prosthesis chip 11. Then the measuring device 20 transmits a measured signal, which is generated according to the induced electrical potential, to the tuning device 30 by a wired or wireless manner

When the signal receiving and transmitting module 21 performs signal transmission in a wired manner, the measuring device 20 can selectively include a transmission line 22 as shown in FIG. 3, so that the signal receiving and transmitting module 21 can transmit the measured signal via the transmission line 22 to the tuning device 30 which will be described later. In one embodiment, if the induced electrical potential from the retinal prosthesis chip 11 detected by the measuring device 20 located on the cornea is too weak, a signal amplifying and filtering device can be further disposed between the tuning device 30 and the measuring device 20. The signal amplifying and filtering device comprises a signal amplifier and a filter for improving the signal quality and intensity of the electrical signal for the tuning device 30 to perform operations. Alternatively, the signal receiving and transmitting module 21 can also transmit the measured signal to the tuning device 30 in a wireless manner like an electrical induction, and the transmission line 22 can be excluded. The present invention has no particular limitation on this.

The tuning device 30 performs an equalization process on the measured signal then sends a calibration setting command and tuning parameters to the transmitting device 40 after the measured signal is processed. In one embodiment of the present invention, the equalization process could be performed based on either a machine learning algorithm or a preset algorithm so as to achieve an automatic equalization process. The equalization process is used to improve the uniformity of the electro-stimulation signals on each of retina neuron cells. In other word, the distribution of the electro-stimulation signals received by each of the retina neuron cells will be more uniform after the equalization process. In one embodiment, each of the pixel units may store a preset parameter for generating the electro-stimulation signal to the retina neuron cells. After receiving the tuning parameters, the preset parameter may be either adjusted based on the tuning parameters or replaced with the tuning parameters.

An electrical induction between the transmitting device 40 and the first induction coil 12 of the retinal implant device 10 allows the first induction coil 12 to receive a required electric power and command (or signal). After the transmitting device 40 receives the calibration setting command and the tuning parameters from the tuning device 30, the transmitting device 40 will transmit a calibration signal that is associated with the calibration setting command and the tuning parameters to the retinal implant device 10. In one embodiment of the present invention, the calibration signal comprises the calibration setting command and the tuning parameters.

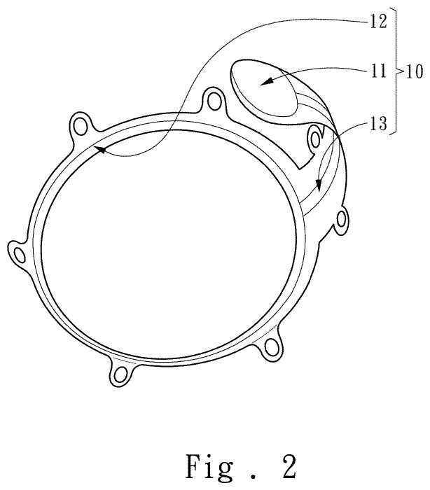

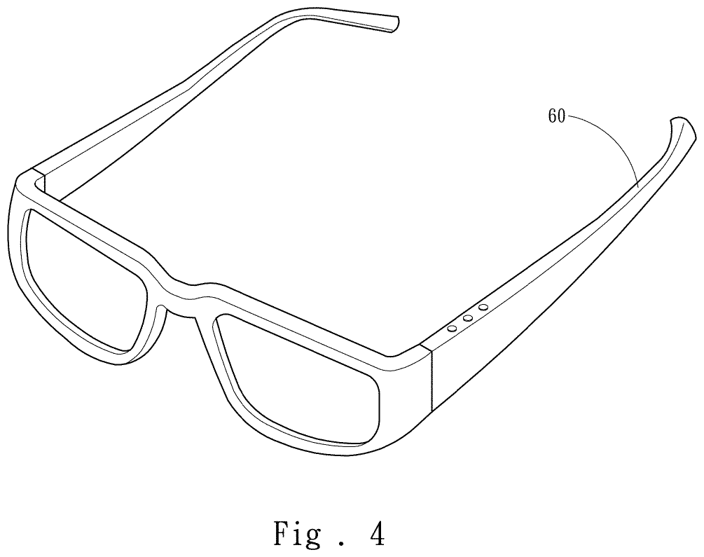

For a specific embodiment of the transmitting device 40, please refer to FIG. 4, which can be designed as a pair of spectacle or goggle 60, and a second induction coil is disposed therein to transmit and receive signals. Referring to FIG. 1 and FIG. 2, in the retinal implant device 10, one end of the electrical connection portion 13 is connected to the first induction coil 12, and another end is connected to the retinal prosthesis chip 11. An electrical induction is formed between the first induction coil 12 and the second induction coil inside the goggles 60. In this embodiment, the transmitting device 40 can supply the retinal prosthesis chip 11 with the required electric power in addition to transmit the calibration signal to the retinal implant device 10. In this way, when the retinal prosthesis chip 11 needs to be charged, the second induction coil in the transmitting device 40 can transmit the electric power to the first induction coil 12 through a wireless charging technique, and then the electric power is supplied to the retinal prosthesis chip 11 via the electrical connection portion 13 to achieve supplying the electric power for the retinal prosthesis chip 11. On the other hand, during the equalization process of the retinal prosthesis system of the present invention, the calibration signal sent by the transmitting device 40 is also transmitted to the first induction coil 12 through the electrical induction, and then transmitted to the retinal prosthesis chip 11 through the electrical connection portion 13 such that the pixel units could adjust the electro-stimulation signal sent to the retina neuron cell based on the tuning parameters. In one embodiment of the present invention, the retinal prosthesis chip 11 further comprises a storage element storing the tuning parameters.

The electro-stimulation signals for stimulating the retina neuron cell generated by the pixel unit (or the electrode) is based on a preset parameter. The parameters of generating the electro-stimulation signal for stimulating the retina neuron cell should be slightly or greatly adjusted for individual pixel unit to achieve better vision perception because of several factors that may cause the less effective stimulation delivery between the pixel unit and the retina neuron cell. Namely, the parameters should be varying with the pixel units.

For example, the factors may comprise the orientation mismatch of the retinal prosthesis chip 11 (or pixel units), the structural defect of the pixel units, the inherent or intrinsic difference between the pixel units, the non-uniform interface between the pixel unit and the retina neuron cell, the shape of eyeball, the characteristic of the retina neuron cell of the user and so on. The measured signal may indicate the mapping or non-uniformity of the electro-stimulation signal sent from the pixel unit or received by the retina neuron cell. Then the equalization process performed by the tuning device 30 could define a suitable tuning parameter (for example, weighting parameter), corresponding to an individual pixel unit or a group of pixel units, for adjusting the electro-stimulation signal by either increasing or decreasing its intensity or duration. In some conventional retina implant device, the preset parameters could be adjusted. However, the adjustment is based on the subject's perceptual responses to the electro-stimulation signal rather than the measurement result from hardware like the present invention. The measurement of the induced electrical potential and tuning of the parameter does not require obtaining the responses from the patient, so the equalization could be done automatically and quickly.

In conventional retinal implant device, a fitting process will be performed to achieve better vision perception of the subject, which is carried out based on the subject's perceptual responses to the electro-stimulation signal. Thus, it is more advantageous to perform the equalization process proposed by the present invention prior to or followed by the fitting process.

In other embodiments, the first induction coil 12 and the electrical connection portion 13 can be excluded, and the relevant components are integrated into the retinal prosthesis chip 11, so that the transmitting device 40 performs signal transmission or power supply directly for the retinal prosthesis chip 11 by a wireless means.

The retinal prosthesis system of the present invention will be explained with the following practical test example. In this example, the retinal prosthesis chip 11 with 4000 pixel units is implanted on a retina of a Lanyu mini pig, and the measuring device 20 is disposed on corneas of the pigs respectively. The measuring device 20 comprises the signal receiving and transmitting module 21 as shown in FIG. 3, and the signal receiving and transmitting module 21 comprises an electrode, such as an electroretinogram (ERG) corneal electrode.

After stimulating the retinal prosthesis system with a light with an illuminance of 400 Lux, the pixel units disposed on the retinal prosthesis chip 11 generate an induced voltage of a biphasic current injection after receiving the light stimulation, the induced voltage of the biphasic current injection is received by the signal receiving and transmitting module 21 of the measuring device 20, and the signal receiving and transmitting module 21 transmits the measured signal to the tuning device 30 via the transmission line 22. In this embodiment, the measuring device 20 obtaining the induced voltage of the biphasic current injection is used as an example, but the present invention is not limited thereto. In other embodiments, it can be other forms of the induced electrical potential (which can be current or voltage) related to the stimulation sent by the pixel units. After the tuning device 30 performs the equalization process on the measured signal, the tuning device 30 sends the calibration setting command and the tuning parameters. After the calibration setting command and the tuning parameters are transmitted to the transmitting device 40, the calibration signal is transmitted by the transmitting device 40 , which is associated with the calibration setting command and the tuning parameters. In one embodiment, the calibration signal is a signal comprising the calibration setting command and the tuning parameters. The calibration signal is then received by the first induction coil 12 and transmitted to the retinal prosthesis chip 11 through the electrical connection portion 13. The tuning parameter for each of the pixel units may be stored in the electronic register, so that the pixel units of the retinal prosthesis chip 11 respectively tune an intensity or a duration of the stimulus according to the tuning parameter, thereby achieving the effect of equalization of the pixel units.

In the above process, the wireless transmission between the transmitting device 40 and the retinal implant device 10 can be performed by using amplitude shift keying (ASK) modulation for transmitting an input command, and by using load shift keying (LSK) modulation as a means for data transmission. The above ASK/LSK signal transmission methods allow energy and data to be transmitted through a same radio frequency electromagnetic wave.

During the test, values detected by the ERG corneal electrodes are continuously recorded. When the values detected by the ERG corneal electrodes are not in a preset range, the light stimulation is repeated to cause the electronic registers of the retinal implant device 10 to tune continuously until the detected values are in the preset range, which means that the equalization process of the retinal prosthesis system is complete. In one embodiment, each of the pixel units sequentially generates the electro-stimulation signal, and at the same time, the measuring device 20 sequentially reads each of the electro-stimulation signals correspondingly, and after the tuning device 30 obtains all of the measured signals, a program of the tuning device 30 calculates a tuning parameter suitable for each of the pixel units, and the tuning parameters are input into the retinal prosthesis chip 11 by the transmitting device 40.

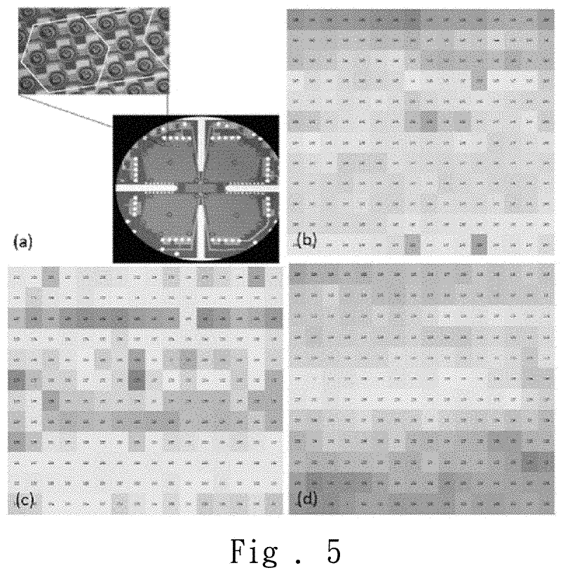

The upper left diagram (a) of FIG. 5 represents the configuration of the 4000 pixel units (divided into 192 groups) on the retinal prosthesis chip 11, and diagrams (b), (c) and (d) are records of group potential/standard deviation of 2.45 mV/0.029 mV, 2.62 mV/0.038 mV, and 2.09 mV/0.061 mV, respectively, wherein some of the pixel units show small variation values. At this point, the equalization process of the present invention will be triggered to tune the retinal prosthesis system. Therefore, at the first time the retinal prosthesis installed, or at any using time after wearing, the retinal prosthesis system of the present invention can perform the equalization process by the above methods without any need for performing active intervention tuning manually, so that the user can always maintain comfortable feeling of clear sense of sight when wearing the retinal prosthesis.

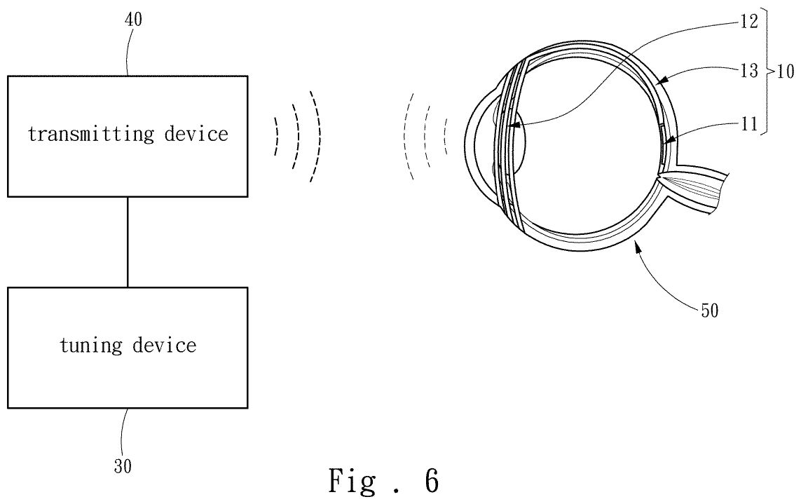

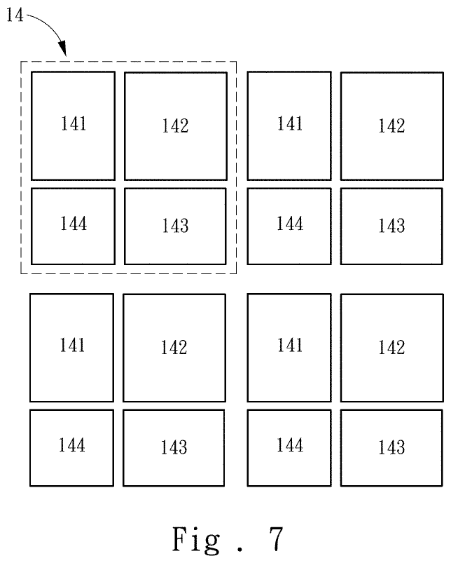

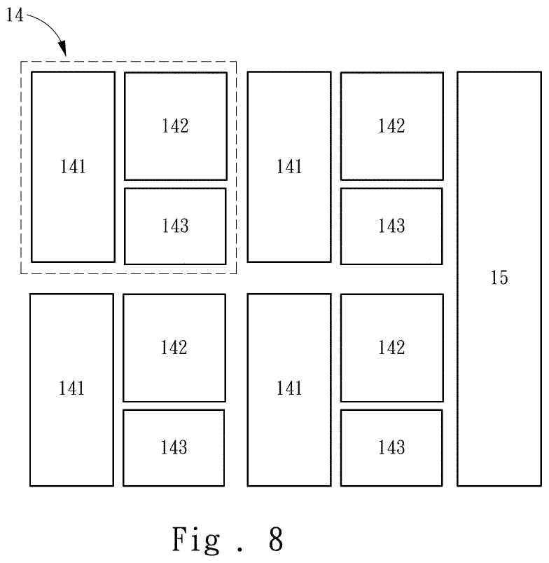

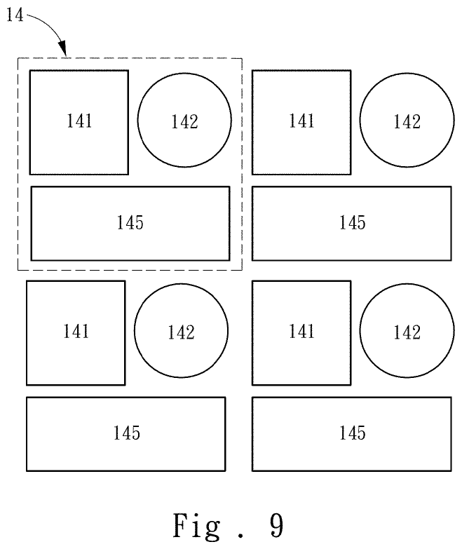

Referring to FIG. 6, another embodiment of the invention relates to a retinal prosthesis system comprising a retinal implant device 10, a tuning device 30, and a transmitting device 40. The retinal implant device 10 includes a retinal prosthesis chip 11, a first induction coil 12, an electrical connection portion 13 and a plurality of pixel units 14 integrated on the retinal prosthesis chip 11. FIG. 7 is a schematic diagram of the pixel unit according to an embodiment of the present invention, each of the pixel units 14 comprises a photosensor 141, an electrode 142, a processing circuitry 143 and an electronic register 144. The electrode 142 is treated as a combination of a stimulator and a measuring component. FIG. 8 is a schematic diagram of the pixel unit according to another embodiment of the present invention, the retinal implant device 10 further comprises a memory bank 15. So there is no need to have the electronic register on the pixel unit. In other embodiments, the electronic register 144 and the memory bank 15 may be replaced with alternative storage element. Further, the processing circuitry 143 can be replaced with a sensing/driving circuitry 145, as shown in FIG. 9.

In one embodiment, the plurality of pixel units 14 receives a light stimulus to generate a light-induced electro-stimulation signal. In an alternative embodiment, the plurality of pixel units 14 receives pixel settings representing a light stimulus to generate a electro-stimulation signal. In another embodiment, the plurality of pixel units 14 receives a command stimulus to generate an electro-stimulation signal. In a further embodiment, the plurality of pixel units 14 receives a command stimulus together data to generate an electro-stimulation signal.

The measuring component measures the electro-stimulation signal outputted from at least one of the plurality of pixel units 14 and transmits a measured signal associated with the plurality of pixel units 14 through the transmitting component. Specifically, the measuring component measures or senses an induced electrical potential of the electro-stimulation signal from the stimulator so as to output the measured signal according to the induced electrical potential. In this embodiment, the first induction coil 12 and the electrical connection portion 13 can be treated as the transmitting component to achieve the communication or electrical connection between the retinal implant device 10 and external parts such as the tuning device 30 and the transmitting device 40.

The tuning device 30 receives the measured signal and performs an equalization process on the measured signal then sends a calibration setting command and tuning parameters. The transmitting device 40 transmits a calibration signal that is associated the calibration setting command and the tuning parameters to the transmitting component. In one embodiment, the plurality of pixel units and the at least one measuring component are integrated in a retinal prosthesis chip disposed on a sub-retina. In another embodiment, the plurality of pixel units, the at least one measuring component and the transmitting component are integrated in a retinal prosthesis chip disposed on a sub-retina.

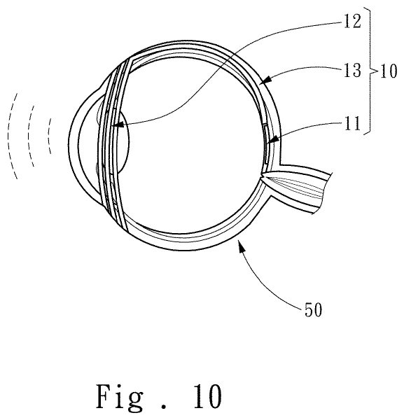

FIG. 10 is a schematic diagram of a retinal implant device according to a further embodiment of the present invention. In this embodiment, the equalization process on the measured signal is carried out by a circuitry in the pixel units. For example, the processing circuitry 143 in FIG. 7. Hence, there is no need for the external measuring device and tuning device.

What is claimed is:

1. A retinal prosthesis system, comprising:
a retinal implant device, comprising a plurality of pixel units to generate an electro-stimulation signal to stimulate retina neuron cells;
a measuring device, measuring an induced electrical potential of the electro-stimulation signal from the retinal implant device and sending a measured signal associated with the induced electrical potential of the plurality of pixel units;
a tuning device, electrically connecting to the measuring device, the tuning device receiving the measured signal and performing an equalization process on the measured signal then sending a calibration setting command and tuning parameters according to the equalization process; and
a transmitting device, transmitting a calibration signal to the retinal implant device, wherein the calibration signal is associated with the calibration setting command and the tuning parameters such that a uniformity of the electro-stimulation signals output from a group of the pixel units is improved.

2. The retinal prosthesis system as claimed in claim 1, wherein the electro-stimulation signal is generated according to a light stimulus received by the plurality of pixel units.

3. The retinal prosthesis system as claimed in claim 1, wherein the electro-stimulation signal is generated according to a command received by the plurality of pixel units.

4. The retinal prosthesis system as claimed in claim 1, wherein the retinal implant device further comprising a first induction coil and an electrical connection portion electrically connected to the pixel units.

5. The retinal prosthesis system as claimed in claim 1, wherein each of the plurality of pixel units is correspondingly disposed with a storage element storing the tuning parameters.

6. The retinal prosthesis system as claimed in claim 1, wherein the retinal implant device further comprises a memory bank.

7. A retinal prosthesis system, comprising:
a retinal implant device, the retinal implant device comprising a plurality of pixel units, at least one measuring component, and a transmitting component, the plurality of pixel units generating an electro-stimulation signal to stimulate retina neuron cell, the at least one measuring component measuring an induced electrical potential of the electro-stimulation signal from the pixel units and sending a measured signal associated with the induced electrical potential of the plurality of pixel units through the transmitting component;
a tuning device, receiving the measured signal and performing an equalization process on the measured signal then sending a calibration setting command and tuning parameters according to the equalization process; and
a transmitting device, transmitting a calibration signal to the retinal implant device, wherein the calibration signal is associated with the calibration setting command and the tuning parameters such that a uniformity of the electro-stimulation signals output from a group of the pixel units is improved.

8. The retinal prosthesis system as claimed in claim 7, wherein the electro-stimulation signal is generated according to a light stimulus received by the plurality of pixel units.

9. The retinal prosthesis system as claimed in claim 7, wherein the electro-stimulation signal is generated according to a command received by the plurality of pixel units.

10. The retinal prosthesis system as claimed in claim 7, wherein each of the plurality of pixel units is correspondingly disposed with a storage element storing the tuning parameters.

11. The retinal prosthesis system as claimed in claim 7, wherein the retinal implant device further comprises a memory bank.

12. The retinal prosthesis system as claimed in claim 7, wherein the plurality of pixel units and the at least one measuring component are integrated in a retinal prosthesis chip disposed on a sub-retina.

13. The retinal prosthesis system as claimed in claim 7, wherein the plurality of pixel units, the at least one measuring component and the transmitting component are integrated in a retinal prosthesis chip disposed on a sub-retina.

14. A retinal implant device, comprising:
a plurality of pixel units, generating an electro-stimulation signal to stimulate retina neuron cell;
at least one measuring component, measuring an induced electrical potential of the electro-stimulation signal from the pixel units and sending a measured signal associated with the induced electrical potential of the plurality of pixel units; and
at least one tuning component, receiving the measured signal and performing an equalization process on the measured signal then generating a calibration setting command and tuning parameters according to the equalization process, wherein a calibration signal associated with the calibration setting command and the tuning parameters is sent to the pixel units such that a uniformity of the electro-stimulation signals output from a group of the pixel units is improved.

15. The retinal implant device as claimed in claim 14, wherein the electro-stimulation signal is generated according to a light stimulus received by the plurality of pixel units.

16. The retinal implant device as claimed in claim 14, wherein the electro-stimulation signal is generated according to a command received by the plurality of pixel units.
